(19) United States
(12) Patent Application Publication
(10) Pub. No.: US 2013/0131629 A1
(43) Pub. Date: May 23, 2013

(54) NANOCHANNELED DEVICE AND RELATED METHODS

(71) Applicants: Alessandro Grattoni, Houston, TX (US); Aman P. Mann, La Jolla, CA (US); Sharath Hosali, Austin, TX (US); Randy Goodall, Austin, TX (US); Lee Hudson, Elgin, TX (US)

(72) Inventors: Alessandro Grattoni, Houston, TX (US); Aman P. Mann, La Jolla, CA (US); Sharath Hosali, Austin, TX (US); Randy Goodall, Austin, TX (US); Lee Hudson, Elgin, TX (US)

(73) Assignee: The Board of Regents of the Unversity of Texas System, Austin, TX (US)

(21) Appl. No.: 13/698,926

(22) PCT Filed: May 19, 2011
(86) PCT No.: PCT/US11/37123

§ 371 Date: Jan. 30, 2013

Related U.S. Application Data
(60) Provisional application No. 61/346,379, filed on May 19, 2010.

(51) Int. Cl. A61M 37/00

(52) U.S. Cl. CPC A61M 37/00
USPC 604/500; 604/93.01; 604/257

(57) ABSTRACT

Apparatus and methods of delivering a therapeutic agent using an implant comprising a nanochannel delivery device.

### CROSS-REFERENCE TO RELATED APPLICATIONS

The present application is a national phase application under 35 U.S.C. §371 of International Application No. PCT/US2011/037123, filed May 19, 2011, which claims priority to U.S. Provisional Application Ser. No. 61/346,379, filed May 19, 2010, and entitled “Nanochanneled Device and Related Methods.” Each of these applications is incorporated herein by reference.

### BACKGROUND INFORMATION

This invention was made with government support under contract NNJ06HE06A awarded by NASA. The government has certain rights in this invention.

Considerable advances have been made in the field of therapeutic agent (e.g. drug) delivery technology over the last three decades, resulting in many breakthroughs in clinical medicine. The creation of therapeutic agent delivery devices that are capable of delivering therapeutic agents in controlled ways is still a challenge. One of the major requirements for an implantable drug delivery device is controlled release of therapeutic agents, ranging from small drug molecules to larger biological molecules. It is particularly desirable to achieve a continuous passive drug release profile consistent with zero order kinetics whereby the concentration of drug in the bloodstream remains constant throughout an extended delivery period.

These devices have the potential to improve therapeutic efficacy, diminish potentially life-threatening side effects, improve patient compliance, minimize the intervention of healthcare personnel, reduce the duration of hospital stays, and decrease the diversion of regulated drugs to abusive uses.

Nanochannel delivery devices may be used in drug delivery products for the effective administration of drugs. In addition, nanochannel delivery devices can be used in other applications where controlled release of a substance over time is needed.

### SUMMARY

In certain embodiments, a nanochannel delivery device may be part of a larger structure configured for implantation into a region or particular area of the human anatomy. For example, nanochannel delivery devices may be a component in an implant configured for a specific orthopedic application. In other embodiments, a nanochannel delivery device may be configured for implantation into an eye. In still other embodiments, a nanochannel delivery device may be part of an apparatus comprising a reservoir with a therapeutic agent, as well as a conduit to deliver the therapeutic agent at a location remote from the reservoir.

In the following, the term “coupled” is defined as connected, although not necessarily directly, and not necessarily mechanically.

The use of the word “a” or “an” when used in conjunction with the term “comprising” in the claims and/or the specification may mean “one,” but it is also consistent with the meaning of “one or more” or “at least one.” The term “about” means, in general, the stated value plus or minus 5%. The use of the term “or” in the claims is used to mean “and/or” unless explicitly indicated to refer to alternatives only or the alternative are mutually exclusive, although the disclosure supports a definition that refers to only alternatives and “and/or.”

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a method or device that “comprises,” “has,” “includes” or “contains” one or more steps or elements, possesses those one or more steps or elements, but is not limited to possessing only those one or more elements. Likewise, a step of a method or an element of a device that “comprises,” “has,” “includes” or “contains” one or more features, possesses those one or more features, but is not limited to possessing only those one or more features. Furthermore, a device or structure that is configured in a certain way is configured in at least that way, but may also be configured in ways that are not listed.

The term “nanochannel delivery device” as used herein comprises any of the exemplary nanochannel devices disclosed in U.S. patent application Ser. No. 12/618,233 filed Nov. 13, 2009 and entitled “Nanochanneled Device and Related Methods” and International Patent Application Number PCT/US10/30937 filed Apr. 13, 2010 and entitled “Nanochanneled Device and Method of Use”, both of which are incorporated herein by reference.

The term “inlet microchannel” is defined as a microchannel through which a molecule travels prior to entering a nanochannel in a nanochanneled delivery device.

The term “outlet microchannel” is defined as a microchannel through which a molecule travels immediately prior to exiting a nanochanneled delivery device.

The term “nanochannel” is defined as a channel with a cross-section having at least one dimension (e.g. height, width, diameter, etc.) that is less than 200 nm.

The term “macrochannel” is defined as a channel with a cross-section having a maximum dimension (e.g. height, width, diameter, etc.) that is greater than about 10 μm.

Certain embodiments comprise an apparatus configured to deliver a therapeutic agent, where the apparatus comprises: an orthopedic implant; a reservoir; and a nanochannel delivery device in fluid communication with the reservoir. In specific embodiments, the orthopedic implant can be configured for implantation into one of the bone group consisting of: femur, tibia, maxillofacial, shoulder, humerus, radius, ulna, wrist, ankle, hip, knee, or spine. In particular embodiments, the orthopedic implant may comprise a cage structure. In specific embodiments, the cage structure is configured to surround a sponge.

In certain embodiments, the reservoir may comprise a therapeutic agent. In particular embodiments, the nanochannel delivery device may be configured to control the release of the therapeutic agent from the reservoir. The reservoir may comprise one or more of the following: an antibiotic, analgesic, anti-inflammatory compound, or growth factor. In specific embodiments, the reservoir may comprise Bone Morphogenetic Protein. In particular embodiments, the apparatus may comprise a protective member configured to protect the nanochannel delivery device from contact with the surrounding environment. In certain embodiments, the protective member can be configured as a screen with apertures.

Particular embodiments may comprise an apparatus configured to deliver a therapeutic agent, where the apparatus comprises: a nanochannel delivery device, where the nanochannel delivery device comprises a plurality of macrochannels, microchannels and nanochannels; and where the macrochannels are configured to form a reservoir containing the therapeutic agent.

In certain embodiments, the nanochannel delivery device may be configured for implantation in a human eye. In specific embodiments, the nanochannel delivery device may be approximately 2 mm wide, 2 mm long, and 0.5 mm thick.

Particular embodiments may comprise an apparatus configured to deliver a therapeutic agent, where the apparatus comprises: a capsule reservoir; a conduit coupled to the reservoir; and a nanochannel delivery device in fluid communication with the conduit. Specific embodiments may further comprise a coupling member coupling the conduit to the capsule reservoir. In particular embodiments the capsule reservoir and the conduit may be integral. In certain embodiments, the nanochannel delivery device may be located within the capsule reservoir. In particular embodiments, the nanochannel delivery device may be located within the conduit. In specific embodiments, the nanochannel delivery device is located within the conduit and proximal to the capsule reservoir.

In particular embodiments, the nanochannel delivery device may be located within a central region of the conduit. In specific embodiments, the nanochannel delivery device may be located within the conduit and proximal to an end of the conduit that is distal to the capsule reservoir. In certain embodiments, the nanochannel delivery device may be located within the conduit and perpendicular to a primary axis of the conduit. In particular embodiments, the nanochannel delivery device may be angled within the conduit.

In specific embodiments, the nanochannel delivery device may be located within the capsule reservoir. The conduit may comprise an upstream portion between the nanochannel delivery device and the reservoir in some embodiments. In particular embodiments, the conduit may comprise a downstream portion between the nanochannel delivery device and a distal end of the conduit, and the upstream portion may comprise a thicker cross-sectional wall than the downstream portion.

Certain embodiments may comprise a method of delivery a therapeutic agent, where the method comprises: providing an implant comprising a reservoir and a nanochannel delivery device, wherein the reservoir comprises the therapeutic agent; inserting the implant into an area of a human or animal anatomy; and releasing the therapeutic agent into the area of the human or animal anatomy. In particular embodiments, the nanochannel delivery device may control the release of the therapeutic agent into the area of the human or animal anatomy.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will be apparent to those skilled in the art from this detailed description.

### BRIEF DESCRIPTION OF THE FIGURES

FIG. 1 is a top view of a wafer used in the manufacture of nanochannel delivery devices according to exemplary embodiments.

FIG. 2 is a top view of a nanochannel delivery device according to exemplary embodiments.

FIG. 3 is a perspective view and a section view of an implant according to an exemplary embodiment.

FIG. 4 is a perspective view of an implant according to an exemplary embodiment.

FIG. 5 is a section view of the embodiment of FIG. 4.

FIG. 6 is a side exploded view of the embodiment of FIG. 4.

FIG. 7 is a perspective exploded view of the embodiment of FIG. 4.

FIG. 8 is a perspective exploded section view of the embodiment of FIG. 4.

FIG. 9 is a perspective view of an implant according to an exemplary embodiment.

FIG. 10 is a section view of the embodiment of FIG. 9.

FIG. 11 is a graph that illustrates a plasma drug concentration over time when the drug is administered via traditional intravenous (IV) methods.

FIG. 12 is a graph that illustrates a plasma drug concentration over time when the drug is administered via a drug eluting implant utilizing an NDD.

FIG. 13 is a side view of a capsule according to an exemplary embodiment.

FIG. 14 is a side view of a capsule according to an exemplary embodiment.

FIG. 15 is a side view of a capsule according to an exemplary embodiment.

FIG. 16A is a side view of a capsule according to an exemplary embodiment.

FIG. 16B is a side view of an alternative configuration of the embodiment of FIG. 16A.

### DETAILED DESCRIPTION OF ILLUSTRATIVE EMBODIMENTS

As previously mentioned, the term “nanochannel delivery device” (or “NDD”) as used herein comprises any of the exemplary nanochannel devices disclosed in U.S. patent application Ser. No. 12/618,233 (the “'233 Application”) filed Nov. 13, 2009 and entitled “Nanochanneled Device and Related Methods” and International Patent Application Number PCT/US10/30937 (the “'937 Application”) filed Apr. 13, 2010 and entitled “Nanochanneled Device and Method of Use”, both of which are incorporated herein by reference.

In certain embodiments, a nanochannel delivery device may form part of a larger assembly that may be used to administer therapeutic agents to a patient. For example, the nanochannel delivery device may be coupled to a capsule or a reservoir that contains the therapeutic agents. The nanochannel delivery device may be used to precisely control the diffusion or passage of small amounts of the therapeutic agent to specific locations within the patient.

In specific embodiments, the NDD, capsule and/or reservoir may be configured specifically for a particular area of the anatomy. For example, the devices may be suitably dimensioned for implantation into an orthopedic or prosthetic implant, or into a patient's eye or other confined or isolated space.

Detailed descriptions of exemplary methods of manufacturing an NDD are provided in the '233 Application and the '937 Application. Therefore, only a brief overview of the final stages of an exemplary NDD manufacturing method will be provided here.

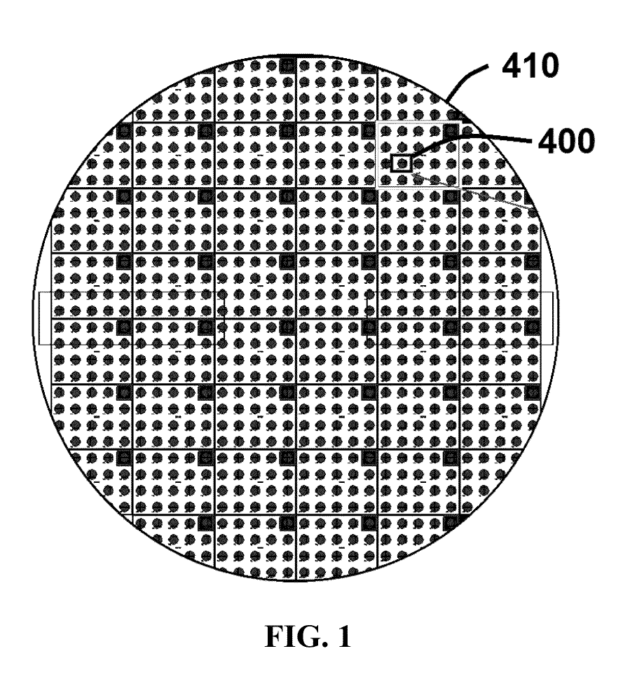

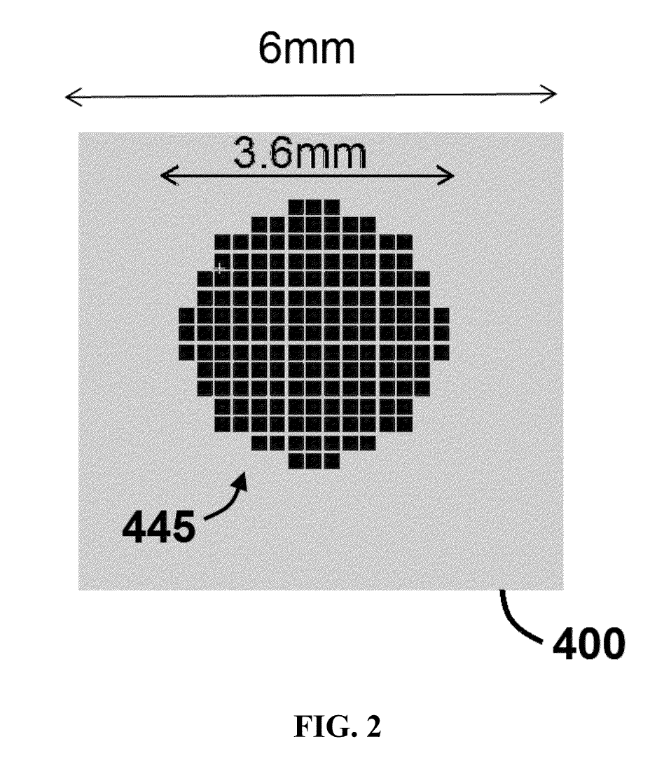

Referring now to FIG. 1, a top view of an entire wafer 410 is illustrated. As shown in this view, wafer 410 (prior to dicing) comprises several nanochannel delivery devices 400 (only one of which is identified in the figure). Wafer 410 can be diced to separate the individual nanochannel delivery devices 400 from each other. A detailed view of an individual nanochannel delivery device 400 with exemplary dimensions is illustrated in FIG. 2. In this view, a plurality of inlet macrochannels 445 are visible on one side of nanochannel delivery device 400. This exemplary embodiment of nanochannel delivery device 400 is approximately 6.0 mm square, and the inlet macrochannels form a generally circular shape approximately 3.6 mm in diameter. It is understood that while wafer 410 of one manufacturing protocol is illustrated in FIG. 1, other protocols will also yield wafers that comprise multiple nanochannel delivery devices, and can be diced or separated into the individual devices. It is also understood that other exemplary embodiments may comprise different dimensions than those shown in FIG. 2.

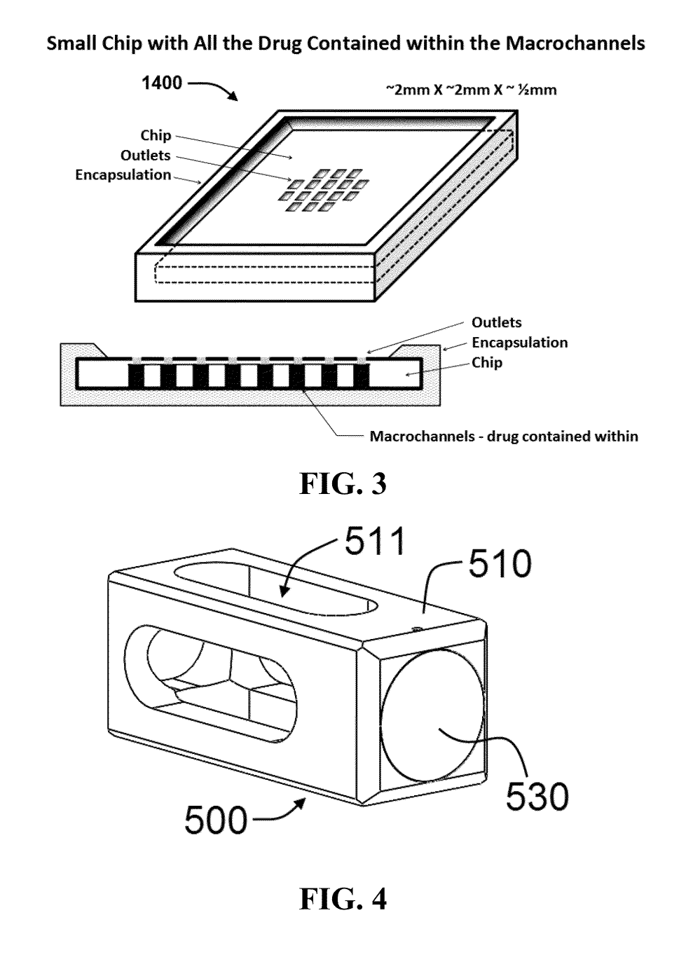

Referring now to FIG. 3, a specific embodiment of a capsule 1400 is shown. This capsule is a minimal covering or encapsulation of the back and sides of the nanochannel device (or “chip”), such that the “reservoir” for a contained drug is limited to the small volume proximal to the openings of the macrochannels on the back of the NDD. The outlets are visible in the non-encapsulated portion of the NDD. At minimum, this space is reduced to be only the macrochannels of the NDD, which offers a volume of about 4.5 cubic millimeters for the embodiment shown in FIGS. 2 and 3.

This embodiment can be made particularly small for implantation in areas with space constraints. In a particular embodiment, the NDD may be approximately 2 mm×2 mm×0.5 mm. Additional volume is possible by fabricating the back surface of the implant such than a thin, planar reservoir is obtained. For example, using the device embodiment of FIG. 2, a planar reservoir in contact with the macrochannels with an internal depth of 1 mm provides 36 μl of volume for drug. To reduce the space occupied by the NDD within the volume of the implant, the chip can be fabricated in a thinned configuration, whereby, after the front side protection layers are applied, the back of the silicon-on-insulator (SOI) wafer is ground and lapped (by methods known in the art) to a thickness between approximately 150 and 500 μm, then further processed as described in the '233 and '937 Applications. These small configurations are especially suited for implantation with very high potency drugs into sensitive locations, e.g., medication into the inner portion of the eye.

In certain embodiments, capsule 1400 may be used to treat Neovascular or exudative age-related macular degeneration (AMD), the “wet” form of advanced AMD. This form of AMD causes vision loss due to abnormal blood vessel growth (choroidal neovascularization) in the choriocapillaris, through Bruch's membrane, ultimately leading to blood and protein leakage below the macula. Bleeding, leaking, and scarring from these blood vessels can eventually cause irreversible damage to the photoreceptors and rapid vision loss if left untreated.

Until recently, no effective treatments were known for wet AMD. However, new drugs, called anti-angiogenics or anti-VEGF (anti-Vascular Endothelial Growth Factor) agents, can cause regression of the abnormal blood vessels and improvement of vision when injected directly into the vitreous humor of the eye. The injections can be painful and frequently have to be repeated on a monthly or bi-monthly basis. Examples of these agents include ranibizumab (trade name Lucentis), bevacizumab (trade name Avastin, a close chemical relative of ranibizumab) and pegaptanib (trade name Macugen). As of April 2007, only ranibizumab and pegaptanib are approved by the FDA for AMD. Bevacizumab is approved, but for other indications. Pegaptanib (Macugen) has been found to have benefits in neovascular AMD. Worldwide, bevacizumab has been used extensively despite its “off label” status. In certain cases, the cost of ranibizumab (Lucentis) is approximately US$2000 per treatment while the cost of bevacizumab (Avastin) is approximately US$150 per treatment.

In certain embodiments, capsule 1400 may be used to administer a compound with anti-angiogenic or anti-vascular endothelial growth factor (VEGF) properties, for example, ranibizumab, bevacizumab, pegaptanib, or other monoclonal antibody or other compound with anti-angeogenic properties, for the treatment of “wet” age-related macular degeneration (AMD). These compounds can prevent the cause of wet AMD, namely abnormal growth of blood vessels under the retina, which can result in damage to or detachment of the retina and loss of vision. Existing methods of administering ranibizumab and pegaptanib include intra-ocular injection every four weeks or six weeks, respectively.

In certain embodiments, an NDD is installed in capsule 1400 as shown in FIG. 3 and the capsule filled (either partially or fully) with a high concentration, e.g., 100 mg/mL bevacizumab solution for use in the treatment of wet AMD. In certain embodiments, the capsule is sized to approximately 30-60 μl, so that the filled capsule contains approximately 3-6 mg of bevacizumab. The capsule can be implanted sub-sclerally and super-choroidally in the front portion of the eyeball through a small incision in a clinical outpatient procedure and removed (with possible replacement) twelve months later through a small incision. Such surgery can be done with conventional techniques. For the smallest possible implant, non-septum filling could be employed, whereby a small hole in the side of the capsule, just larger than the filling needle, is used to inject the bevacizumab into the implant. This small hold is wiped and sealed with a quick setting epoxy. Because the risks associated with accidental impact are lower inside the eye, physical stiffness requirements on the capsule may be lower, allowing thinner walls to be employed. Especially in the case of injection molding, the capsule can be curved in shape to accommodate interior eyeball geometry (radius of curvature approximately 12 mm).

The micro- and nano-channel sizes of the nanochannel delivery device can be chosen (for example, according to the model described in [Grattoni, A. Ferrari, M., Liu, X. Quality control method for micro- nano-channels silicon devices. U.S. Patent Application No. 61/049,287 (April 2008)]), to provide a release rate of about 8 μg/day, which can be maintained for about one year in certain embodiments. In this example, the nanochannel delivery device configuration with this behavior uses a 2.2×2.6 mm chip size, with one macrochannel with opening of 200×600 μm, and within the macrochannel approximately 120 rows of nanochannel structures, consisting of 12 each of inlet and outlet microchannels, connected through about 24 nanochannels according to the description herein. In this embodiment, he inlets and outlets are approximately 1×3 μm in cross-section, with the inlets being about 30 μm long and the outlets being about 1.6 μm long, and the nanochannels are about 3 μm long and 3 μm wide and 30 nm high. Other exemplary configurations with different dimensions that yield approximately the same release rate and duration may be derived from the mathematical model.

In other embodiments, an NDD may be used in conjunction with a reservoir located within a prosthetic or orthopedic implant. One particular embodiment comprises the inclusion of a NDD in an orthopedic implant configured for use in a spine to provide anterior lumbar interbody fusion. Existing anterior lumbar interbody fusion implants may comprise a cage structure surrounding a collagen sponge saturated with a therapeutic agent, e.g., Bone Morphogenetic Protein-2 (BMP-2). The cage is typically a hollow serrated block or screw device which is placed in the disc space between two lumbar vertebrae after the removal of a defective disc.

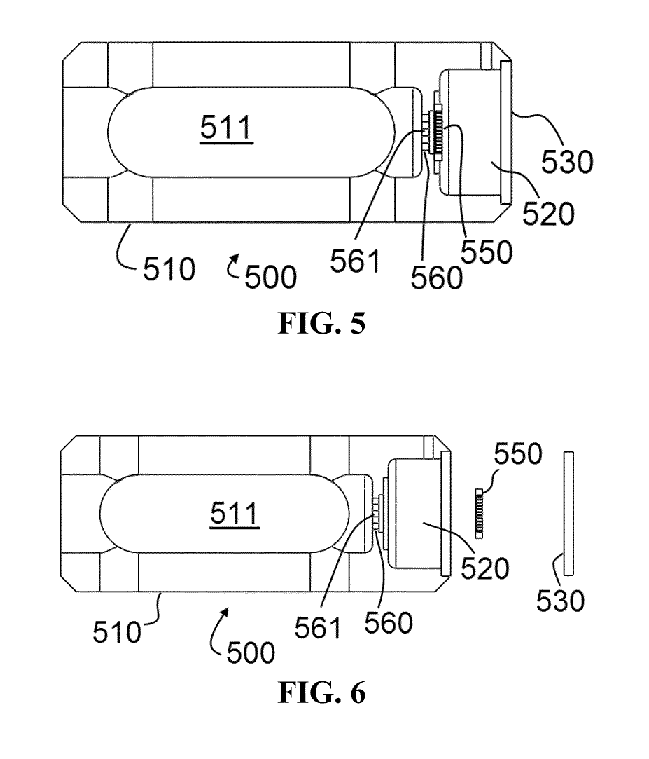

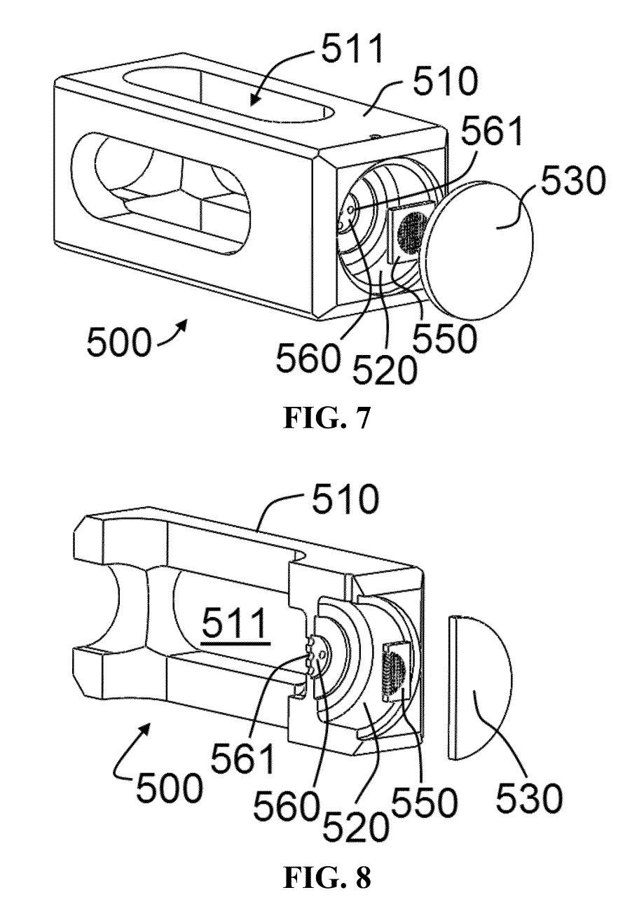

Referring now to FIGS. 4-8, a fusion implant 500 is configured for use in vertebral fusion, e.g. anterior lumbar interbody fusion. Fusion implant 500 comprises a cage structure 510 with an interior space 511. In particular embodiments, interior space 511 may be approximately 8 cubic centimeters (cc) in volume. Cage structure 510 can provide mechanical stability as well as a framework for new bone growth to create vertebral fusion.

In the embodiment shown, fusion implant 500 comprises a reservoir 520 configured to contain a therapeutic agent. Fusion implant 500 may also comprise a cap or cover 530 configured to seal reservoir 520 after a therapeutic agent has been placed in reservoir 520. In the embodiment shown, an NDD 550 is in fluid communication with reservoir 520 and may be used to precisely control the diffusion or passage of the therapeutic agent to the patient. In specific embodiments, the therapeutic agent may be released from reservoir 520 into a substrate (e.g., a collagen sponge, not shown for purposes of clarity) located within cage structure 510.

Fusion implant 500 may also comprise a protective member configured to prevent contact between NDD 550 and the surrounding environment, and to serve as a barrier between bodily fluids and NDD 550. In certain embodiments, the protective member may be configured as a screen 560 with a plurality of apertures 561. Screen 560 should be configured to allow the therapeutic agent to pass through screen 560 to the tissue surrounding fusion implant 500. During use, the therapeutic agent can be released from reservoir 520 through NDD 550 and apertures 561 of screen 560 into the surrounding environment.

In exemplary embodiments, apertures 561 are configured so that they do not restrict the diffusion of the therapeutic agent from reservoir 520 into the surrounding tissue. Reservoir 520 is configured so that the therapeutic agent is directed to go through NDD 550 before the therapeutic agent can exit reservoir 520 into the surrounding tissue. The diffusion rate is therefore controlled by NDD 550 based on the configuration of NDD 550 (e.g., the dimensions and quantity of nanochannels and microchannels in NDD 550).

In certain embodiments, the therapeutic agent may comprise Bone Morphogenetic Protein, including Bone Morphogenetic Protein-2 (BMP-2) and Bone Morphogenetic Protein-7 (BMP-7). In one exemplary embodiment, reservoir 520 comprises a volume of 0.25 cc and is configured to deliver BMP-2 over a particular period of time. In a specific embodiment, NDD 550 may be configured to deliver 0.1-1.0 milligrams (mg) of BMP-2 over 3-4 weeks. In other specific embodiments, NDD 550 may be configured to deliver 400 micrograms (g) of BMP-2 per day for 30 days, or 200 g per day for 60 days, or 133 g per day for 90 days.

In a particular exemplary embodiment, NDD 550 may comprise a nanochannel height (as defined in the '233 Application and the '937 Application) of approximately 20 nanometers (nm). It is calculated that 22,932 nanochannels would be needed (with microchannel dimensions of 1 m by 8 m with a nanochannel length of 1 m) in order to achieve delivery of 400 g of BMP-2 per day.

In other exemplary embodiments, fusion implant 500 may be configured to deliver therapeutic agents such as antibiotics in the prophylactic treatment of local infections. In a specific embodiment, fusion implant 500 is configured to deliver Cefazolin, an antibiotic that is used after orthopedic surgery. Cefazolin is often delivered by intravenous injection over several days. In specific embodiments utilizing fusion implant 500, two grams of Cefazolin may be placed in reservoir 520 and delivered at the rate of 200 mg per day for 10 days of local prophylactic treatment. With a concentration of 2 g/ml in a 1 cc reservoir, it is calculated that 284,004 nanochannels would be needed (with microchannel dimensions of 1 m by 8 m with a nanochannel length of 1 m) in order to achieve delivery of 200 mg of Cefazolin per day.

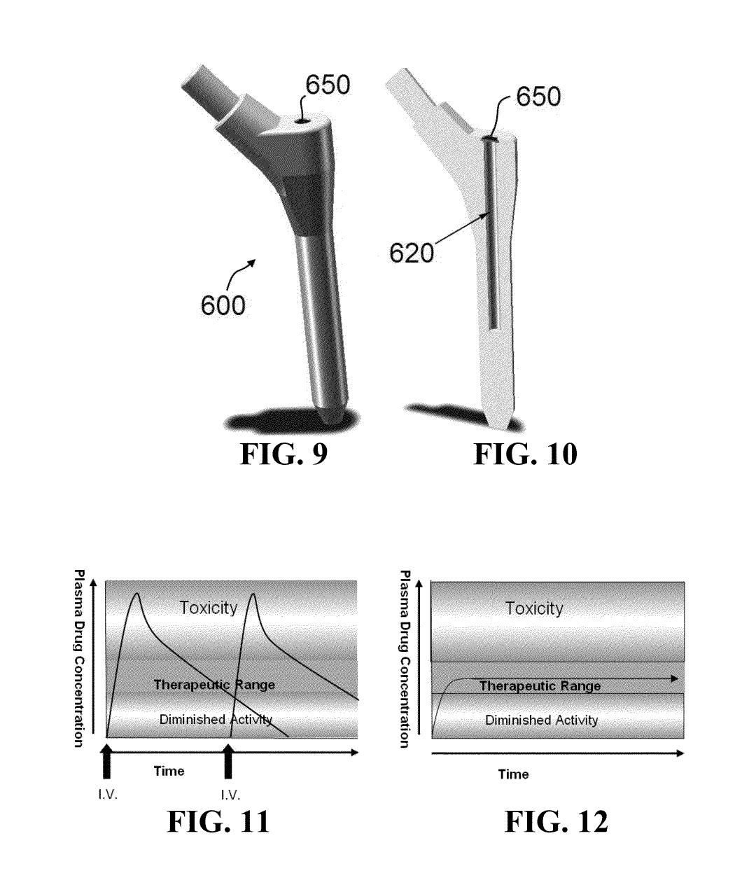

In other embodiments, an NDD may be used in conjunction with a reservoir located within a prosthetic or orthopedic implant including, for example, a femoral implant. Referring now to FIGS. 9 and 10, one exemplary embodiment comprises a femur implant 600 comprising a reservoir 620 and an NDD 650. Similar to the previously described embodiments, reservoir 620 may contain one or more therapeutic agents, the delivery of which is controlled via NDD 650 (which is in fluid communication with reservoir 620).

Typical physical post surgical issues after fitting a prosthesis include adequate wound healing, pain management and inflammatory response. Additional medication in the form of antibiotics, antithrombotics, as well as growth factors (e.g. tissue and bone regeneration factors) typically need to be supplemented post surgery. In certain embodiments, reservoir 620 may contain therapeutic agents, including, for example, antibiotics, analgesics, and anti-inflammatory compounds in order to address such issues. In certain embodiments, reservoir 620 may comprise a protective screen (not shown) similar to screen 560 in the previously-described embodiment. The therapeutic agent or agents are delivered from reservoir 620, through NDD 650 into the articular space around the joint.

In exemplary embodiments, implants according to the present disclosure may be constructed from a biocompatible material, e.g. silicone, ceramics, polymer, or polyvinychloride (PVC), or polyether ether ketone (PEEK). In certain embodiments, implants according to the present disclosure may be metals including, for example, titanium, stainless steel or Nitinol.

While a femur implant has been shown in FIGS. 9 and 10, other orthopedic implants comprising an NDD may be configured for implantation into other bones, e.g. a femur, tibia, maxillofacial, shoulder, humerus, radius, ulna, wrist, ankle, hip, knee, or spine. Such bones are typically large enough that an implant can be sized to accommodate a reservoir and NDD, while still maintaining the required structural rigidity.

In certain embodiments, an implant may comprise multiple reservoirs filled with different therapeutic agents to be released. For example, antibiotics, analgesics, antithrombotics (to prevent blood coagulation), anti-inflammatory agents (to counter the acute inflammation) may be released along with agents configured for tissue and bone regeneration factors. An NDD with appropriately selected release characteristics may be selected to control the agent release of each reservoir.

Exemplary embodiments provide benefits associated with a sustained release or delivery of the therapeutic agent. For example, a drug eluting implant utilizing an NDD to control the release of a therapeutic agent can lead to a quicker and a more comfortable recovery. Referring now to FIG. 11, a graph illustrates a plasma drug concentration over time when the drug is administered via traditional intravenous (IV) methods. As illustrated the concentration of the drug is initially above the therapeutic range, and then lowers over time into the therapeutic range, and finally falls below the therapeutic range. When the concentration falls below the therapeutic range, an additional IV dosage is administered and the process repeats itself. As shown in FIG. 11, the fluctuating concentration resulting from this method of administration does not allow the concentration to be maintained in the therapeutic range consistently in the therapeutic range. This can result in longer recovery times and additional discomfort to the patient.

Referring now to FIG. 12, a graph illustrates a plasma drug concentration over time when the drug is administered via a drug eluting implant utilizing an NDD. As illustrated, the concentration can be maintained more consistently due to the NDD providing a controlled release of the drug or therapeutic agent from the implant. As shown in FIGS. 11 and 12, compared to routine administration of drug in plasma, a lower amount of therapeutic agent can be administered and a higher local concentration can be achieved utilizing an implant with an NDD. This can result in lower side effects and provide a minimally invasive way of delivering drugs.

In certain embodiments, it may be desirable to have the reservoir for the therapeutic agent located remotely from the point at which it is administered.

In certain embodiments, a capsule may comprise a primary capsule reservoir (PCR) and a capsule extension (CE) or conduit. The conduit allows the therapeutic agent which diffuses through the NDD (exemplary embodiments of which are described in the '233 and the '937 Applications) to exit the capsule via the conduit and first encounter body tissue at a distance from the PCR. Such a configuration may be beneficial in certain environments. For example, the volume of the therapeutic agent needed may require a capsule larger than the in vivo space available, such as in a bone joint. In such cases, there may be too little blood flow to the area where the therapeutic agent is needed to allow for effective intravenous delivery. The PCR and conduit combination can provide for larger volumes of a therapeutic agent to be delivered remotely.

In exemplary embodiments, the conduit should be filled, without entrapped bubbles, with a fluid that acts as an extended diffusion medium for the exiting molecules, providing a continuous fluid path from inside the PCR, through the nanochannels, through the conduit, and into the body fluid at the distal end of the conduit. The NDD may be internal to the PCR or within the conduit, being located proximal, medial, or distal to the PCR.

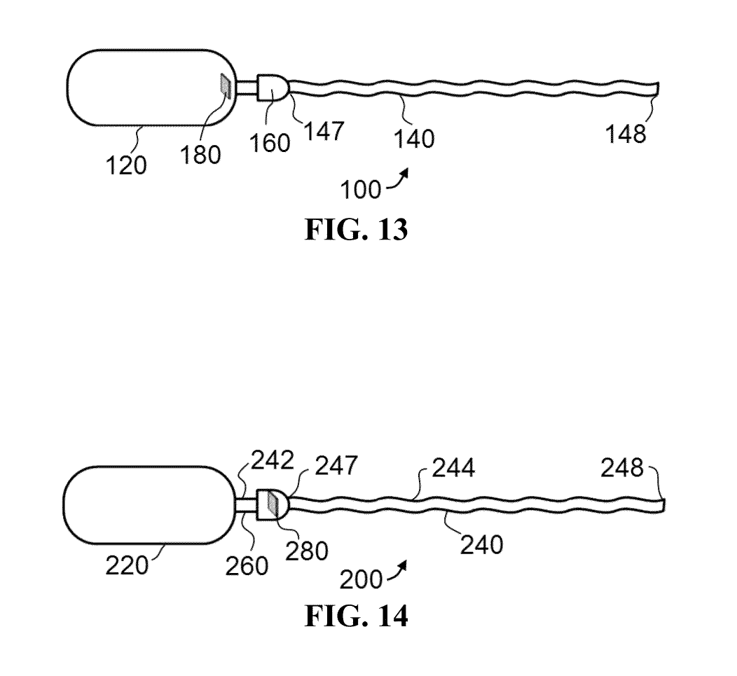

Referring now to FIG. 13, a first exemplary embodiment of a capsule 100 comprises a PCR 120 and a conduit 140 coupled via a coupling member 160. In other embodiments, conduit 140 may be directly coupled to PCR 120 (e.g., via insertion into an aperture in PCR 120, or forming conduit 140 and PCR 120 as an integral unit). Conduit 140 comprises a proximal end 147 and a distal end 148.

In this embodiment, an NDD 180 is located within PCR 120. The diffusion of therapeutic molecules from PCR 120 is controlled via NDD 180 as described in the '233 Application. In exemplary embodiments, conduit 140 may be any suitable implantable tubing with an inner cross sectional area not substantially less than the sum of the areas of all the outlet microchannels of NDD 180, for example, greater than 1 mm for NDD 180 shown in FIG. 13. In exemplary embodiments, conduit 140 does not have significant effective release characteristics; it merely remotely deposits already-released molecules. In certain embodiments, therapeutic molecules may not be released until they reach distal end 142 of conduit 140. In other embodiments, conduit 140 may comprise a plurality of apertures along all or part of its length to release therapeutic molecules.

Conduit 140 may be flexible or rigid depending on the application requirements. The length of conduit 140 may also vary depending on the application requirements. In certain embodiments, conduit 140 may be 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 cm long.

Referring now to FIG. 14, a capsule 200 is configured similar to capsule 100. For example, capsule 200 comprises a PCR 220 coupled to a conduit 240 via a coupling member 260. In this embodiment, however, an NDD 280 is located within conduit 240 but proximal to PCR 220. In certain embodiments, NDD 280 may be located within approximately 1-30 millimeters of PCR 20. In the particular embodiment shown, NDD 280 is located within coupling member 260. In other embodiments, NDD 280 may be located in a portion of conduit 240 that is proximal to PCR 220, but not within a coupling member.

The diffusion of therapeutic molecules from PCR 220 is controlled via NDD 280 as in previously-described embodiments. In this embodiment, conduit 240 may be usefully a separate fabricated component that attaches to PCR 220 either temporarily or permanently. Because conduit 240 contains NDD 280, PCR 220 can remain constant, while conduit 240 controls the release performance. In addition, by configuring conduit 240 as separate and detachable, conduit 240 can remain in place if PCR 220 needs to be repaired, replaced, and/or refilled. In this exemplary embodiment, an upstream portion 242 of conduit 240 (e.g., a portion of conduit 240 between PCR 220 and conduit 240) is comprises thicker walls to prevent excessive bending that could significantly change the interior volume of conduit 240 (thereby expressing the contents of PCR 220 through NDD 280). A downstream portion 244 of conduit 240 comprises a proximal end 247 and a distal end 248 and may be any suitable material as described in capsule 100 above. In exemplary embodiments, upstream portion 242 may comprise a thicker cross-sectional wall than downstream portion 244.

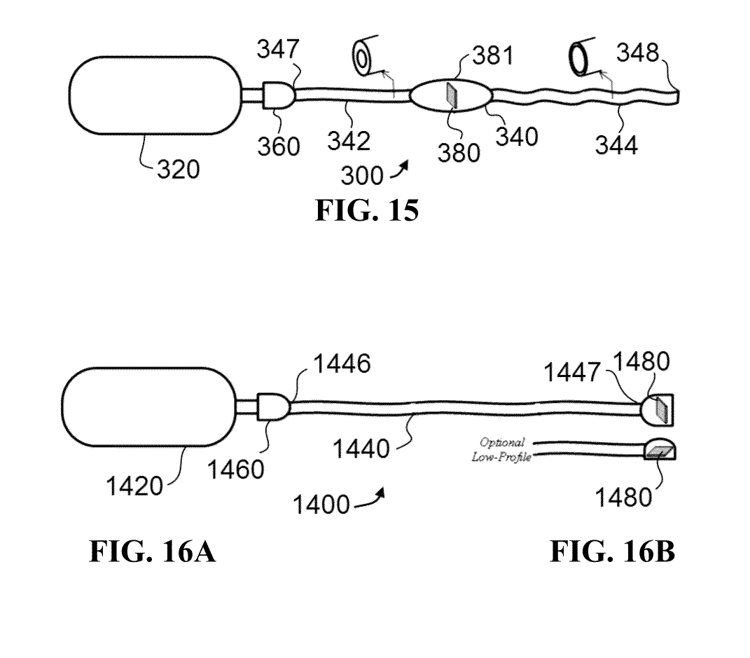

Referring now to FIG. 15, a capsule 300 comprises a PCR 320 coupled to a conduit 340 via an optional coupling member 360. Capsule 300 is configured similar to capsule 200 but with an NDD 380 located within and near a central region of conduit 340 (e.g. in the region approximately half way between PCR 320 and a distal end 348 of conduit 340 that is distal from PCR 320). In certain embodiments, NDD 380 may be located within an optional housing 381 coupled to an upstream portion 340 and a downstream portion 344 of conduit 340. As shown in the partial cross-section views of upstream and downstream portions 340, 344, the wall thickness of upstream portion 340 may be thicker than that of downstream portion 344. This configuration can provide rigidity between a proximal end 347 of conduit 340 and NDD 380, and allow flexibility downstream of NDD 380.

The operational characteristics and therapeutic molecule diffusion control are similar to that described above for capsule 200. The configuration provided in capsule 300 can be useful when a short rigid upstream extension is desired for moving through an active tissue layer, for example, the abdominal wall, that could constrict a flexible tube during normal physical activity, but allows for the final downstream extension to be flexible.

Referring now to FIG. 16A, a capsule 1400 comprises a PCR 420 coupled to a conduit 1440 via an optional coupling member 1460 at a proximal end 1447 of conduit 1440. In this embodiment, an NDD 1480 is located within conduit 1440 and at or near a distal end 1448 of conduit 1440 distal from PCR 1420. The operational characteristics and therapeutic molecule diffusion control are similar to that described above for capsules 200 and 300. This configuration can be useful when a concern exists about the ability of the tissues to maintain a continuous fluid path within conduit 1440. In this case, NDD 480 is in close contact with the target tissue. In this embodiment, all of conduit 1440 should be somewhat rigid because it is all “upstream” of NDD 1480. This configuration facilitates guided installation of PCR 1420 and conduit 1440 into the body “by feel” (e.g. without direct visualization). NDD 1480 may face the end of conduit 1440 (e.g., be arranged generally perpendicular to the primary axis of conduit 1440). In the embodiment shown in FIG. 16B, NDD 1480 is angled within conduit 1440 to provide a lower profile (e.g. cross-section) of conduit 1440 to ease insertion and withdrawal.

### REFERENCES

The contents of the following references are incorporated by reference herein:
[1] Santen, R. J., Yue, W., Naftolin, F., Mor, G., Berstein, L. The potential of aromatase inhibitors in breast cancer prevention. Endocrine-Related Cancer. 6, 235-243 (1999).
[2] Goss, P. E., Strasser, K. Aromatase Inhibitors in the Treatment and Prevention of Breast Cancer. J. Clin. Oncol. 19, 881-894 (2001).
[3] Chlebowski, R. T. Reducing the Risk of Breast Cancer. N. Engl. J. Med., 343, 191-198 (2000).
[4] Dowsett, M., Jones, A., Johnston, S. R., Jacobs, S., Trunet, P., Smith, I. E. In vivo measurement of aromatase inhibition by letrozole (CGS 20267) in postmenopausal patients with breast cancer. Clin. Cancer Res. 1, 1511-1515 (1995).
[5] Brueggemeier, R. W., Hackett, J. C., Diaz-Cruz, E. S. Aromatase Inhibitors in the Treatment of Breast Cancer. Endocrine Reviews 26, 331-345 (2005).
[6] Coates, A. S., Keshaviah, A., Thürlimann, B., et al. Five years of letrozole compared with tamoxifen as initial adjuvant therapy for postmenopausal women with endocrine-responsive early breast cancer: update of study BIG 1-98. J. Clin. Oncol. 25, 486-492 (2007).
[7] Goss, P. E., Ingle, J. N., Martino, S., et al. A randomized trial of letrozole in postmenopausal women after five years of tamoxifen therapy for early-stage breast cancer. N. Engl. J. Med. 349, 1793-1802 (2003).
[8] Garreau, J. R., Delamelena, T., Walts, D., Karamlou, K., Johnson, N. Side effects of aromatase inhibitors versus tamoxifen: the patients' perspective. Am. J. Surg. 192, 496-8 (2006).
[9] Luthra, R., Kirma, N., Jones, J., Tekmal, R. R. Use of letrozole as a chemopreventive agent in aromatase overexpressing transgenic mice. The Journal of Steroid Biochemistry and Molecular Biology. 86, 461-467 (2003).
[10] Harper-Wynne, C., Ross, G., Sacks, N., Salter, J., Nasiri, N., Iqbal, J., A'Hern, R., Dowsett, M. Effects of the aromatase inhibitor letrozole on normal breast epithelial cell proliferation and metabolic indices in postmenopausal women: a pilot study for breast cancer prevention. Cancer Epidemiol. Biomarkers Prev. 11,614-21 (2002).
[11] Walczak, R. J. et al. Long-Term Biocompatibility of NanoGATE Drug Delivery Implant. Nanobiothech. 1, 35-42, (2005).
[12] Martin, F. et al. Tailoring width of microfabricated nanochannels to solute size can be used to control diffusion kinetics. J. Controlled Release. 102, 123-133, (2005).
[13] Pricl, S. et al. Multiscale modeling of protein transport in silicon membrane nanochannels. Part 1. Derivation of molecular parameters from computer simulations. Biomed. Microdev. 8, 277-290 (2006).
[14] Cosentino, C. et al. Multiscale modeling of protein transport in silicon membrane nanochannels. Part 2. From molecular parameters to a predictive continuum diffusion model Biomed. Microdev. 8, 291-298 (2006)
[15] Smolensky, M. H., Peppas, N. A. Chronobiology, drug delivery, and chronotherapeutics. Adv. Drug Deliv. Rev. 59, 828-851, (2007).

1. An apparatus configured to deliver a therapeutic agent, the apparatus comprising:
an orthopedic implant;
a reservoir; and
a nanochannel delivery device in fluid communication with the reservoir.

2. The apparatus of claim 1, wherein the orthopedic implant is configured for implantation into one of the bone group consisting of: femur, tibia, maxillofacial, shoulder, humerus, radius, ulna, wrist, ankle, hip, knee, or spine.

3. The apparatus of claim 1, wherein the orthopedic implant comprises a cage structure.

4. The apparatus of claim 3, wherein the cage structure is configured to surround a sponge.

5. The apparatus of claim 1 wherein the reservoir comprises a therapeutic agent.

6. The apparatus of claim 5 wherein the nanochannel delivery device is configured to control the release of the therapeutic agent from the reservoir.

7. The apparatus of claim 1 wherein the reservoir comprises one or more of the following: an antibiotic, analgesic, anti-inflammatory compound, or growth factor.

8. The apparatus of claim 1 wherein the reservoir comprises Bone Morphogenetic Protein.

9. The apparatus of claim 1 wherein the apparatus comprises a protective member configured to protect the nanochannel delivery device from contact with the surrounding environment.

10. The apparatus of claim 9 wherein the protective member is configured as a screen with apertures.

11. An apparatus configured to deliver a therapeutic agent, the apparatus comprising:
a nanochannel delivery device, wherein the nanochannel delivery device comprises a plurality of macrochannels, microchannels and nanochannels;
wherein the macrochannels are configured to form a reservoir containing the therapeutic agent.

12. The apparatus of claim 11 wherein the nanochannel delivery device is configured for implantation in a human eye.

13. The apparatus of claim 11 wherein the nanochannel delivery device is approximately 2 mm wide, 2 mm long, and 0.5 mm thick.

14. An apparatus configured to deliver a therapeutic agent, the apparatus comprising:
a capsule reservoir;
a conduit coupled to the reservoir; and
a nanochannel delivery device in fluid communication with the conduit.

15. The apparatus of claim 14 further comprising a coupling member coupling the conduit to the capsule reservoir.

16. The apparatus of claim 14 wherein the capsule reservoir and the conduit are integral.

17. The apparatus of claim 14 wherein the nanochannel delivery device is located within the capsule reservoir.

18. The apparatus of claim 14 wherein the nanochannel delivery device is located within the conduit.

19. The apparatus of claim 14 wherein the nanochannel delivery device is located within the conduit and proximal to the capsule reservoir.

20. The apparatus of claim 14 wherein the nanochannel delivery device is located within a central region of the conduit.

21. The apparatus of claim 14 wherein the nanochannel delivery device is located within the conduit and proximal to an end of the conduit that is distal to the capsule reservoir.

22. The apparatus of claim 14 wherein the nanochannel delivery device is located within the conduit and perpendicular to a primary axis of the conduit.

23. The apparatus of claim 14 wherein the nanochannel delivery device is angled within the conduit.

24. The apparatus of claim 14 wherein:
the nanochannel delivery device is located within the capsule reservoir;
the conduit comprises an upstream portion between the nanochannel delivery device and the reservoir;
the conduit comprises a downstream portion between the nanochannel delivery device and a distal end of the conduit; and
the upstream portion comprises a thicker cross-sectional wall than the downstream portion.

25. A method of delivery a therapeutic agent, the method comprising:
providing an implant comprising a reservoir and a nanochannel delivery device, wherein the reservoir comprises the therapeutic agent;
inserting the implant into an area of a human or animal anatomy; and
releasing the therapeutic agent into the area of the human or animal anatomy.

26. The method of claim 25 wherein the nanochannel delivery device controls the release of the therapeutic agent into the area of the human or animal anatomy.
